(19) United States
(12) Patent Application Publication
(10) Pub. No.: US 2019/0296987 A1
(43) Pub. Date: Sep. 26, 2019

(54) SYSTEM AND METHOD FOR ENHANCING PATIENT COMMUNICATION

(71) Applicants: Tom COTTON, Cheshire, CT (US); Daniel YASOSHIMA, North Haledone, NY (US); Jeren KOH, Bristol, CT (US)

(72) Inventors: Tom COTTON, Cheshire, CT (US); Daniel YASOSHIMA, North Haledone, NY (US); Jeren KOH, Bristol, CT (US)

(21) Appl. No.: 16/364,249

(22) Filed: Mar. 26, 2019

Related U.S. Application Data
(60) Provisional application No. 62/823,169, filed on Mar. 25, 2019.
(60) Provisional application No. 62/719,353, filed on Aug. 17, 2018.
(60) Provisional application No. 62/648,211, filed on Mar. 26, 2018.

(51) Int. Cl. H04L 12/24; G06F 3/0481; H04L 29/08; G06F 3/01; G06F 9/451; G16H 80/00

(52) U.S. Cl. CPC H04L 41/22; G06F 3/0481; H04L 67/12; G16H 80/00; G06F 3/012; G06F 9/451; H04L 67/26

(57) ABSTRACT

The system for improving communication between a user and a caregiver within an environment such as a hospital includes a wireless communication network facilitating communication between a caregiver communication device and a user communication device. A customizable graphic user interface is displayed on a user communication device and displays GUI features available for selection by user input. Upon selection of a GUI feature by the user, a notification is sent to the caregiver communication device, alerting the caregiver of the need to attend to the user, e.g., a nurse to a patient. The GUI includes a graphical cursor that can be manipulated via user head tracking, enabling hands-free selection of GUI features and notification of caregivers by the users. The movements of the caregivers are tracked via the caregiver communication devices to register when a caregiver is in the proximity of the user to attended to the user request. The status of user requests are monitored and the response time for caregivers to the user requests are recorded to ensure sufficient attentiveness to the user.

### CROSS REFERENCE TO RELATED APPLICATION(S)

This application claims the benefit of U.S. Provisional Patent Application Nos. 62/823,169, filed Mar. 25, 2019; 62/719,353, filed Aug. 17, 2018; and 62/648,211, filed Mar. 26, 2018, which are incorporated by reference as if disclosed herein in their entirety.

### BACKGROUND

Patient care systems are generally known. These patient care systems assist patients in communicating with caregivers. Conventional patient care systems allow patients to “page” their caregivers and request assistance, e.g., a call bell system. However, it is problematic when conventional communication systems do not allow patients to communicate their needs to a caregiver with specificity, or when a patient has a disability or other physical impediment that prevents them from using a conventional communication device. It is further problematic when patients have difficulty communicating with their caregivers because they do not speak the same language and the conventional communication device they are using does not have a feature that compensates for this language barrier. As a result, communication between patients and their caregivers becomes inefficient, resulting in a delay between recognition of a need by the patient, e.g., pain management, and actual intervention by the caregiver. Current systems also typically utilize physical input from users in order to notify their caregiver. However, such systems may not be useable for physically impaired or otherwise recuperating patients. Patients with one or more IVs also display hindered movement, and it is highly common for IVs to become dislodged merely while attempting to notify a caregiver, producing two needs where originally there was just one.

From the caregiver's perspective, these call bell systems merely provide generic indicators that some intervention is needed. Without additional information, however, the caregiver is unable to prioritize requests, which may result in a delayed response to a more urgent request while attending another lower urgency request.

### SUMMARY

Methods and systems according to the disclosed subject matter are directed to a system for improving communication between a user and a caregiver within an environment including a wireless communication network, a caregiver communication device in the environment, a graphic user interface (GUI) displayed on a user communication device in the environment in communication with the caregiver communication device, the GUI of the user communication device including a graphical cursor configured to identify a location of user input in the GUI, a database of GUI features available for display in the GUI and for selection in the GUI by the user input, and a non-transitory computer storage media coupled with the user communication device and encoded with one or more computer programs for facilitating communication of the user communication device with the caregiver communication device. In some embodiments, the system includes one or more user-family communication devices, wherein the notification module is configured to transmit a notification to the one or more user-family communication devices of one or more GUI features selected by the user.

In some embodiments, the one or more computer programs includes a GUI customization module configured to organize the GUI features in the GUI for presentation to a user via the user communication device and selection by the user via the graphical cursor

In some embodiments, the one or more computer programs include a notification module configured to transmit a notification to the caregiver communication device of one or more GUI features selected by the user and transmit notifications from the caregiver to the user communication device. In some embodiments, the notification module includes a messaging module configured to transmit non-GUI feature messages between the caregiver communication device and the user communication device. In some embodiments, the notification module transmits notifications from the user communication device and the caregiver communication device to an electronic medical record for the user.

In some embodiments, the one or more computer programs include a hands-free module, the hands-free module being in communication with the GUI and configured to enable manipulation of the graphical cursor within the GUI without physical contact with the user communication device by the user. In some embodiments, the hands-free module enables manipulation of the graphical cursor via facial recognition, head-tracking, voice recognition, or combinations thereof.

In some embodiments, the hands-free module enables selection of a first GUI feature via predetermined overlap duration of the graphical cursor and the first GUI feature in the GUI, and the hands-free module includes a head-tracking module in communication with the GUI, the head-tracking module being configured to manipulate the graphical cursor within the GUI in response to movement of a user's head. In some embodiments, the head-tracking module is configured to continuously capture frames corresponding to an environment in front of the user communication device, identifying the presence of a user's face within the frames, define one or more reference points on the user's face, position a virtual window enclosing the one or more reference points, the virtual window corresponding to the graphical cursor, monitor the position of the one or more reference points at a time (t) compared to a prior time (t−x), determine a flow vector for a reference point corresponding to movement of the reference point from the prior time (t−x) to time (t), and updating the position of the graphical cursor corresponding to an average of the flow vectors. In some embodiments, the sensitivity of the head-tracking module can be increased or decreased as needed.

In some embodiments, the one or more computer programs include a caregiver tracking module configured to track the location of a caregiver in the environment and identify when the caregiver is in proximity with the user. In some embodiments, the one or more computer programs include a data analytics module configured to record data regarding the caregiver communication device, the user communication device, the GUI features, the transmitted notifications, the hands-free-module, or combinations thereof. In some embodiments, the one or more computer programs include a translation module configured to receive a GUI feature selection in a first language and output an equivalent notification in a second language.

In some embodiments, the GUI features include user-needs identifiers related to general user assistance, user bathroom needs, user present status, user mood, user pain level, user pain location, food requests, user satisfaction, predefined messages, environmental controls, or combinations thereof. In some embodiment, the notifications from the caregiver include informational documents, responses to GUI features selected by the user, presence of the caregiver communication device at a predetermined location within the environment or combinations thereof. In some embodiments, the notifications include a status indicator corresponding to whether the one or more GUI features selected by the user have been addressed. In some embodiments, addressing the one or more GUI features selected by the user includes identifying the caregiver communication device at a predetermined location within the environment via WiFi, global positioning system, near-field communication, radio-frequency identification, or combinations thereof, scanning by the caregiver communication device of a code associated with the one or more GUI features, or combinations thereof.

In some embodiments, the system includes one or more wireless beacons configured to track the location of the caregiver communication device within the environment, wherein the status indicator of the one or more GUI features is changed to completed when the caregiver communication device enters a user's room. In some embodiments, the user communication device is reversibly positioned on a stand. In some embodiments, the stand includes a reversibly extendable arm mounted to a floor, a ceiling, a wall, a wheelchair, a base, or combinations thereof. In some embodiments, movement of the reversibly extendable arm is controlled via the user communication device.

Some embodiments of the present disclosure are directed to a computer implemented method for improving communication between a user and a caregiver. In some embodiments, the method includes providing a caregiver communication device, providing a user communication device in communication with the caregiver communication device, providing a graphic user interface (GUI) displayed on the user communication device, the GUI including a graphical cursor configured to identify a location of user input in the GUI, providing a database of GUI features available for display in the GUI and for selection in the GUI by the user input, displaying one or more GUI features to the user on the user communication device, selecting one or more GUI features on the user communication device by the user, generating a notification to the caregiver communication device of a selection by the user of one or more GUI features, and identifying that the notification has been addressed via proximity of the caregiver communication device to the user communication device.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic drawing of a system for enhancing patient communication according to some embodiments of the present disclosure;

FIG. 2 is a graphical user interface for use in a system for enhancing patient communication according to some embodiments of the present disclosure;

FIG. 3 is a schematic drawing of a system for enhancing patient communication according to some embodiments of the present disclosure;

FIG. 4 is a graphical user interface for use in a system for enhancing patient communication according to some embodiments of the present disclosure;

FIG. 5 is a schematic drawing of a system for enhancing patient communication according to some embodiments of the present disclosure;

FIG. 6A is a chart of a method for enhancing patient communication according to some embodiments of the present disclosure;

FIG. 6B is a chart of a method for enhancing patient communication according to some embodiments of the present disclosure; and

FIG. 6C is a chart of a method for enhancing patient communication according to some embodiments of the present disclosure.

### DETAILED DESCRIPTION

It is recognized that certain limitations and features described in the present disclosure, such as the transmission and viewing of personal medical information, may need to be modified or removed in order to be in compliance with applicable local laws. For example, in the United States, certain limitations and features of the present disclosure may need to be modified or removed in order to be in compliance with the Health Insurance Portability and Accountability Act (HIPAA).

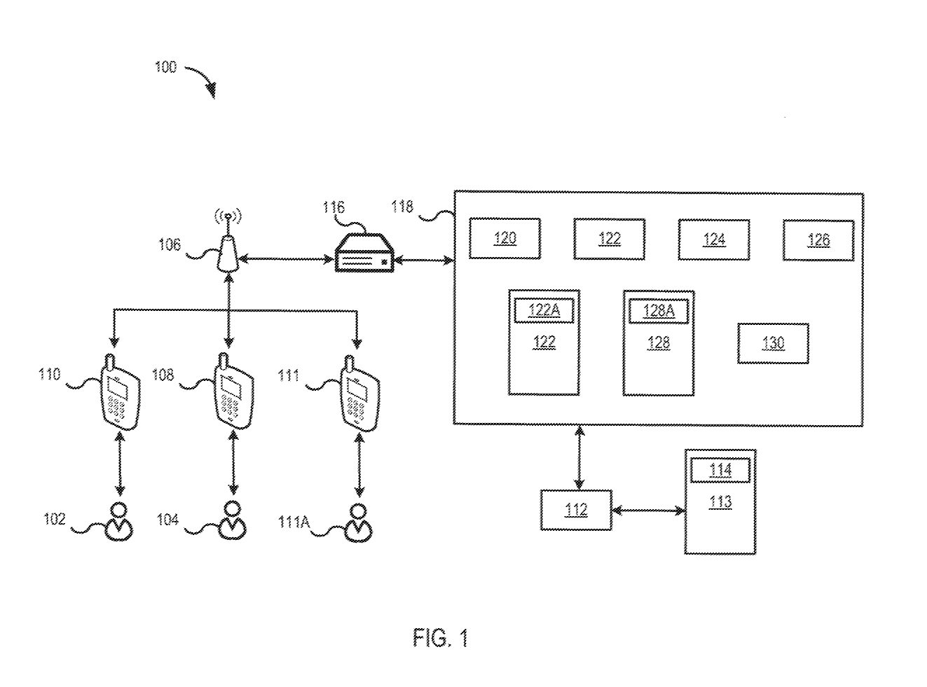

Referring now to FIG. 1, aspects of the disclosed subject matter include system 100 for improving communication between a user 102 and a caregiver 104 within an environment. While the exemplary embodiments of system 100 discussed in the present disclosure are implemented in a healthcare environment, e.g., a hospital, long-term care facility, etc., the environments in which system 100 can be implemented are not limited in this regard, as the environment can be any environment where a user 102 would request service or attention from a caregiver 104, such as on a mode of transportation, e.g., train, airplane, ship, etc., in a restaurant, in a supervised park, e.g., a national park, an amusement park, etc., and the like. In some embodiments, system 100 includes a communication network 106. In some embodiments, communication network 106 is wireless, wired, or combinations thereof. In some embodiments, communication network 106 includes at least one WiFi network, at least one Bluetooth network, at least one local area network, or combinations thereof. In some embodiments, system 100 includes sufficient encryption to allow secure communication of information within communication network 106, e.g., between user 102 and caregiver 104. In some embodiments, individuals using system 100 remain anonymous to other users, or at least are not identified by any personal information. In some embodiments, users of system 100 can create personal profiles to store and maintain preferential settings for the system for current and future usage.

Communication network 106 is configured to allow communication between system 100 and one or more caregiver communication devices 108 and one or more user communication devices 110. In some embodiments, communication network 106 is configured to allow communication between caregiver communication devices 108 and user communication devices 110. System 100 includes sufficient hardware to support and facilitate communication between components of the system as desired, e.g., routers, wireless access points, servers, etc. as will be known to those having ordinary skill in the art. In some embodiments, caregiver communication device 108 and/or user communication device 110 are configured to store data locally, access data stored remotely, or combinations thereof. System 100 also includes sufficient software to support and facilitate communication between components of the system as desired, e.g., data structures, data transfer protocols, data encryptors, data encoders, etc., as will be known to those having ordinary skill in the art. While the exemplary embodiments of system 100 describe communication between one or more caregiver communication devices 108 and one or more user devices 110 for clarity, the communication devices are not limited in this regard, as one or more third-party communication devices 111 can also be in communication with system 100 and its components, e.g., to enable communication with caregiver communication device 108 and one or more user device 110. In some embodiments, third-party communication devices 111 are user-family 111A communication devices, enabling, for example, a patient's family to be advised as to the status of their loved one, e.g., their pain level, satisfaction rating, etc.

In some embodiments, caregiver communication device 108 and user communication device 110 are any suitable electronic computing device for communicating via communication network 106 and enabling the other aspects of the present disclosure. In some embodiments, caregiver communication device 108 and/or user communication device 110 are smartphones, tablet PCs, PDAs, wearables, e.g., smartwatches, laptops PCs, desktop PCs, etc., or combinations thereof. In some embodiments, caregiver communication device 108 and/or user communication device 110 include one or more displays. In some embodiments, the displays are touchscreens, e.g., capacitive, resistive, etc., configured to accept user input. In some embodiments, caregiver communication device 108 and/or user communication device 110 are in communication with an input controller, e.g., a mouse, trackpad, etc., or combinations thereof, configured to accept user input.

In some embodiments, system 100 includes a graphic user interface (GUI) 112 displayed on user communication device 110, e.g., via the display mentioned above. In some embodiments, GUI 112 includes a graphical cursor configured to identify a location of user input in the GUI. In some embodiments, system 100 includes one or more databases 113 of GUI features 114 available for display in GUI 112. In some embodiments, GUI features 114 are available for selection in GUI 112 by user input, e.g., via the input controller. In some embodiments, GUI features 114 displayed in GUI 112 are customizable, as will be discussed in greater detail below. In some embodiments, GUI features 114 displayed in GUI 112 are customized by the user, caregiver, an administrator, or combinations thereof.

In some embodiments, system 100 includes a non-transitory computer storage media 116 coupled with user communication device 110. Computer storage media 116 can be any suitable removable or non-removable media for storing information, e.g., data structures, APIs, etc. In some embodiments, computer storage media includes RAM, ROM, flash memory, CD-ROM, DVD, BLURAY, or combinations thereof. In some embodiments, computer storage media 116 includes a memory, e.g., local hard drive, cloud storage, etc., or combinations thereof.

In some embodiments, computer storage media 116 is encoded with one or more computer programs 118 for facilitating communication of user communication device 110 with the caregiver communication device 108. In some embodiments, computer programs 118 are executed on a processor of caregiver communication device 108 and user communication device 110, via a web browser, etc., or combinations thereof.

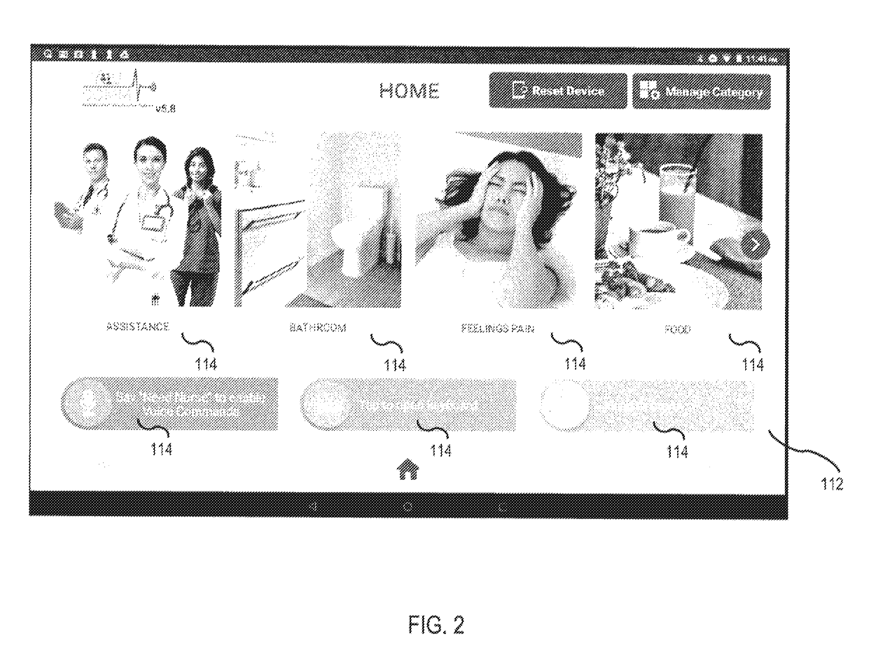

In some embodiments, one or more computer programs include a GUI customization module 120. In some embodiments, GUI customization module 120 is configured to organize GUI features 114 in GUI 112 for presentation to a user, e.g., via user communication device 110. GUI features 114 placed via GUI customization module 120 are then selectable in GUI 112 by user 102, e.g., via the graphical cursor. Referring now to FIG. 2, as discussed above, in some embodiments, system 100 includes databases 113 of GUI features 114 that can be incorporated into GUI 112 to customize GUI layout and functionality according to user preferences and/or needs. In some embodiments, GUI features 114 include user-needs identifiers related to general user assistance, user bathroom needs, user present status, user mood, user pain level, user pain location, food requests, user satisfaction, e.g., overall or in real-time, predefined messages, environmental controls, e.g., controlling lights, televisions, radios, etc., or combinations thereof. In some embodiments, GUI features 114 can include any combination of images, videos, text, audio, or combinations thereof representative of the GUI feature. Upon selection of GUI feature 114 via GUI customization module 120, the selected GUI feature will be displayed on GUI 112 via, e.g., the aforementioned representative images, videos, text, or audio. In some embodiments, any number of GUI features 114 can be included in GUI 112. In some embodiments, GUI features 114 are organized in GUI 112 into one or more menus, e.g., a menu of foods available for order that day.

In some embodiments, GUI features 114 selectable in GUI 112 (and thus on the relevant communication device) are defined by user 102 themselves, i.e., the user customizes GUI 112 with desired GUI features 114. In some embodiments, GUI features 114 selectable in GUI 112 (and thus on the relevant communication device) are defined by caregiver 104 or administrator on the user's behalf, e.g., IT staff, attending physician, responsible nurse, etc.

In general, individuals and/or administrators using system 100 select any number of the GUI features 114 that correspond to communications that user 102 desires or could desire to send to another individual using system 100. In the exemplary embodiment shown in FIG. 2, the user is a patient in a hospital. In this embodiments, GUI customization module 120 provides the patient with a number of GUI features 114 related to patient recovery, treatment, etc., and then displays those GUI features to the patient. The high degree of GUI customization enabled by GUI customization module 120 provides faster and more efficient communication by users 102 regarding their needs. In the exemplary embodiment, GUI features 114 that are irrelevant to the specific patient can be removed from GUI 112, decluttering the interface for improved ease-of-use and decreasing the time user 102 might need to sort through the irrelevant GUI features 114 to find the one that is needed. For example, some patients in a hospital may find it advantageous to have a GUI feature dedicated to calling a nurse to assist in using the restroom, while others may not. Patients needing the restroom-assistance GUI feature can then implement that feature in their customized GUI via GUI customization module 120, while other patients can remove that GUI feature in favor of a cleaner interface or another feature entirely. In some embodiments, GUI 112 includes one or more preset “themes” designed to cater to a specific group. In some embodiments, the presets include themes for children, e.g., friendly background colors, images representing GUI features, etc.; seniors, e.g., simplified menu layouts, larger fonts, etc.; and the like. In some embodiments, the presets provide dedicated spatial positioning and organization of GUI features 114. In some embodiments, the layout or composition of GUI 112 is scheduled to change at a predetermined time, such that GUI features 114 selectable at a first time are replaced with different GUI features 114 at a second time. By way of example, during the morning, GUI 112 can include a food menu including a plurality of GUI features 114 corresponding to the various foods available for breakfast. At a predetermined time, e.g., 11 AM, the GUI features in the food menu can be scheduled to change to include GUI features 114 corresponding to the various foods available for lunch. In some embodiments, daily food special GUI features can be scheduled to accurately reflect the specials available that day.

Referring again to FIG. 1, in some embodiments, system 100 includes a notification module 122. Notification module is configured to transmit communications between at least caregiver communication device 108 and user communication device 110. In some embodiments, the communication is in the form of a notification presented on the display of caregiver communication device 108 or user communication device 110. In some embodiments, the communication is in the form of instructions to computer storage medium 116 to perform some action, e.g., via computer programs 118. In some embodiments, the notification is a notification to caregiver communication device 108 of one or more GUI features 114 selected by a user. In some embodiments, the notification includes text, image, video, audio, or combinations thereof. In some embodiments, the notification includes a representation, e.g., image, video, audio, etc., of the one or more GUI features 114 selected by the user. In some embodiments, notifications include informational documents, responses from caregiver 104 to GUI features selected by user 102, presence indicator for caregiver communication device 108 at a predetermined location within the environment, or combinations thereof. In some embodiments, notification module 122 transmits communications from user communication device 110, caregiver communication device 108, or between at least caregiver communication device 108 and user communication device 110 to an electronic medical record (EMR) for the user. In some embodiments, the system is configured to export the EMR or information included therein. In some embodiments, each selected GUI feature 114 produces an individual notification. In some embodiments, a plurality of selected GUI features 114 can be included in a single notification, e.g., a plurality of GUI features can be selected and then a “Send Requests” GUI feature can be selected to send one notification notifying caregiver communication device 108 of the plurality of selected GUI features 114.

As discussed above, in some embodiments, GUI features 114 include predefined messages. In some embodiments, these predefined messages are stock questions, comments, or commands that can be selected by a user 102 and/or caregiver 104 in order to quickly transmit a communication, e.g., for something particularly important or frequent. By way of example, user 102 can include a GUI feature for a preset message of “Can I please have my medication.” Selection of this preset message in GUI 112 on user communication device 110 then sends the corresponding notification to caregiver communication device 108 so that caregiver 104 is alerted that user 102 needs their medication. In some embodiments, preset messages are displayed on GUI 112 as representative text, images, video, etc., e.g., to improve legibility. By way of example, the preset message “Can I please have my medication” can be represented on GUI 112 as an icon including an image of the medicine itself.

In some embodiments, notification module 122 includes a messaging module 122A configured to transmit messages between caregiver communication device 108 and user communication device 110. In some embodiments, the messages are non-GUI feature messages, e.g., those composed on an in-GUI keyboard or some other messaging input controller, e.g., a physical QWERTY keyboard.

In some embodiments, notifications include a status indicator corresponding to whether the notification has been acted upon, e.g., one or more GUI features 114 selected by user 102 have been addressed, a message from caregiver 104 to user 102 has been responded to, etc. In some embodiments, the time between when a GUI feature 114 is selected by user 102 and caregiver 104 addresses the notification, i.e., the response time, is monitored, measured, and/or recorded. In some embodiments, a selected GUI feature is “addressed” via identifying caregiver communication device 108 at a predetermined location within the environment, scanning by caregiver communication device 108 of a code associated with user communication device 110 upon which the GUI feature was selected, selecting a corresponding “request addressed” GUI feature on user communication device 110, or combinations thereof. In some embodiments, the location of caregiver communication device 108 is monitored via a caregiver tracking module 124. In some embodiments, caregiver tracking module 124 is configured to track the location of caregiver communication device 108, and as a result, caregiver 104, in the environment and identify when the caregiver is in proximity with user 102, e.g., via proximity to user communication device 110, the user's room, furniture with the user's room (a bed/chair), etc., or combinations thereof. In some embodiments, the location of caregiver communication device 108 is determined via WiFi, global positioning system, near-field communication, radio-frequency identification, or combinations thereof. By way of example, user 102 can select on GUI 112 a GUI feature 114 requesting “more water.” A notification is then sent to caregiver communication device 108 indicating the user's request and assigning a status indicator that the request has not yet been addressed. Caregiver 104 gets the water and brings it to user 102. In some embodiments, the status of the request is changed to “addressed” when the caregiver with the caregiver communication device 108 enters the user's room to deliver the water. In some embodiments, the request status is changed when the caregiver scans a code, e.g., a QR code, specific and generated in response to the user request.

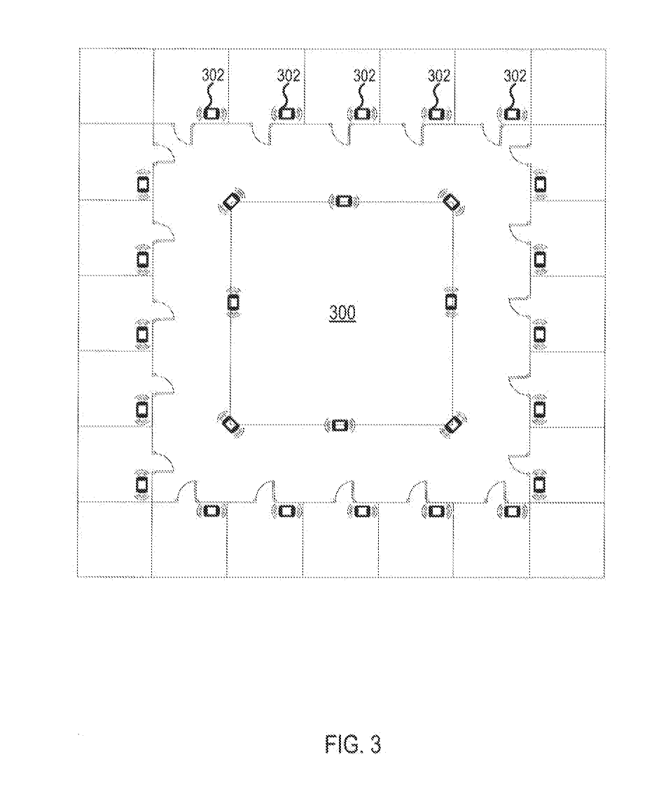

Referring now to FIG. 3, caregiver tracking module 124 includes one or more wireless beacons 302 positioned within an environment 300. In some embodiments, wireless beacons 302 are distributed throughout environment 300. In some embodiments, user communication device 110 is one of wireless beacons 302. Via wireless determination of relative proximity of caregiver communication device 108 to the various beacons 302, the location in and movement through environment 300 can be tracked and recorded. As discussed above, in some embodiments, when caregiver tracking module 124 identifies caregiver communication device 108 within a predetermined proximity of user 102 who generated a request, system 100 can be configured to identify this proximity as an indication that the request is being addressed by caregiver 104 and change the status of the user request to “addressed.”

Referring again to FIG. 1, as discussed above, response time of the caregiver to the user request can be monitored and recorded. In some embodiments, system 100 incentivizes caregivers to achieve lower response times through the availability of incentives for high-performing caregivers, e.g., those caregivers having average response times below a predetermined threshold. In some embodiments, the incentives take the form of tangible rewards, such as prizes, bonuses, etc., or intangible rewards, such as points, a ranking, etc. In some embodiments, caregiver response time is a metric made available to other caregivers to foster a competitive environment. This gamification can be highly beneficial in promoting engagement and improvement in caregivers 104 using system 100.

In some embodiments, system 100 includes a translation module 126. Translation module 126 is configured to receive GUI feature 114 selection notifications from notification module 122 and/or messages from messaging module 122A in a first language and output an equivalent notification in a second language. Translation module 126 therefore greatly simplifies communication from users 102 and caregivers 104 that have issues communicating, e.g., because they fail to speak/understand the same language. By way of example, a patient might select a GUI feature for “Can I please have my medication?” which is translated via translation module 126 such that the notification actually sent to caregiver communication device 108 is “¿Puedo por favor tener mi medicatión?” In some embodiments, translation module 126 also translates notifications and/or messages from caregiver communication device 108 to user communication device 110.

In some embodiments, system 100 includes a hands-free module 128. Hands-free module 128 is in communication with GUI 112 and configured to enable manipulation of the graphical cursor within the GUI without physical contact with a display or other input controller in use with caregiver communication device 108 or user communication device 110. In some embodiments, hands-free module 128 enables manipulation of the graphical cursor via facial recognition, head-tracking, voice recognition, or combinations thereof. In some embodiments, hands-free module enables selection of GUI features 114 via facial recognition, head-tracking, voice recognition, or combinations thereof, as will be discussed in greater detail below.

In some embodiments, hands-free module 128 includes a head-tracking module 128A, the head-tracking module being in communication with the GUI and configured to enable manipulation of the graphical cursor within the GUI via movement of the user's head, e.g., within an environment in front of user communication device 110. In some embodiments, hands-free module continuously captures frames corresponding to an environment in front of the user communication device 110. In some embodiments, hands-free module 128 includes one or more cameras configured to capture frames in the environment in front of user communication device 110. Using suitable image recognition tools, e.g., algorithms, trained networks, or combinations thereof configured to identify objects within an image, the presence of a user's face within the frames is identified. Head-tracking module 128A then defines one or more reference points on the user's face. A virtual window corresponding to the graphical cursor is then generated, where the virtual window encloses the one or more reference points. Head-tracking module 128A continues to monitor the position of the one or more reference points. At each time (t) that the position of the one or more reference points is registered, the current registered location is compared to a registered location of the one or more reference points at a prior time (t−x). If the reference point locations between t and t−x have changed, a flow vector is assigned for the reference point corresponding to the movement of the reference point from the prior time (t−x) to time (t). The position of the graphical cursor is then updated corresponding to an average of the flow vectors. In some embodiments, when the difference between a current registered location and a previous registered location is beyond a certain threshold, the virtual window is reset to a new one or more reference points. In some embodiments, if no new set of reference points can be found by the image recognition algorithms/networks, the virtual window can be completely reset and head-tracking module 128A can begin searching for a new face to track.

In some embodiments, the sensitivity of movement of the graphical cursor in response to the magnitude of the flow vectors is adjustable, enabling user 102 to increase or decrease the response of head-tracking module 128A to movement of their head. In some embodiments, hands-free module 128 enables selection of GUI features 114 via sustained overlap in GUI 112 of the graphical cursor and a GUI feature. In embodiments featuring head-tracking module 128A, for example, “selection” of a GUI feature is a matter of moving the user's head towards the desired GUI feature to move, in kind, the reference window to overlap with the GUI feature. After the reference window has overlayed the GUI feature for a predetermined amount of time, that GUI feature is considered “selected” and a notification can be sent from notification module 122. In embodiments including voice recognition, voice commands are recognized and make selections of GUI features 114 in the same way graphical cursor can select those GUI features. In some embodiments, the voice recognition features make use of CMU Sphinx library algorithms, e.g., a mix of gaussian phoneme models and Markov chains, cloud-based services, or combinations thereof.

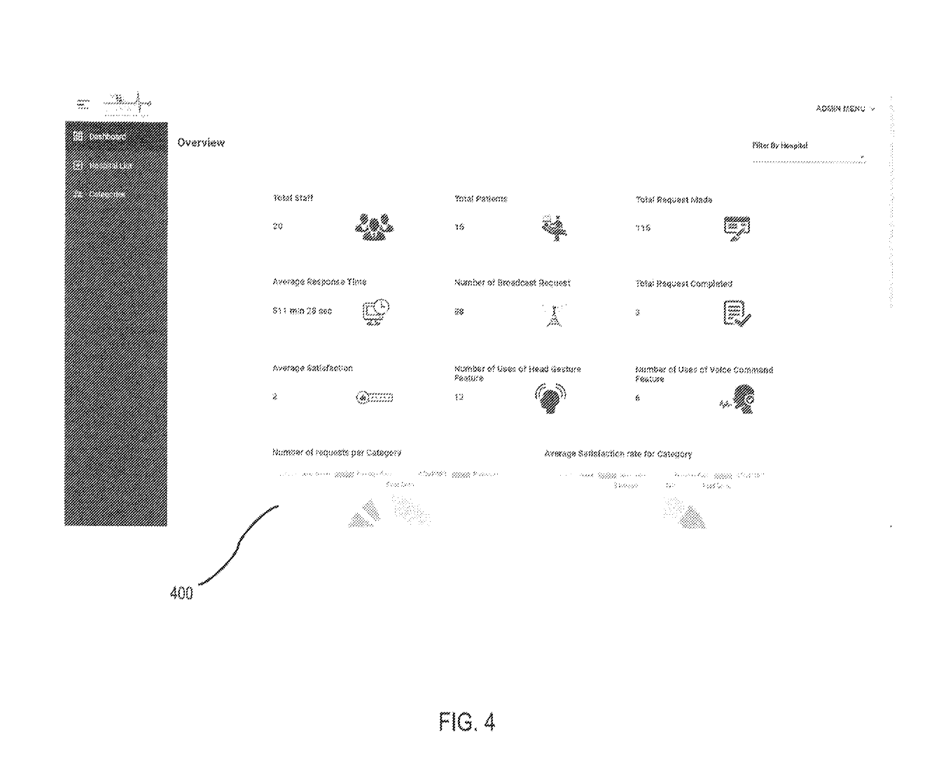

In some embodiments system 100 includes a data analytics module 130. In some embodiments, data analytics module 130 is configured to record data regarding caregiver communication device 108, user communication device 110, third-party device 111, GUI 112, GUI features 114, notifications transmitted from notification module 122, activity of hands-free-module 128, or combinations thereof. Referring now to FIG. 4, in some embodiments, data analytics module 130 enables visualization of the recorded data, e.g., in a report viewable to users, administrators, etc., such via a virtual dashboard 400. In some embodiments, data analytics module 130 enables the setting of custom alarms based on the recorded data. For example, if a user has failed to issue an adequate number of requests for meals, an alarm may trigger to alert one or more caregivers to intervene and ensure the user is getting sufficient nutrients. In some embodiments, data analytics module 130 is effective to manage staffing in the environment. The number and frequency of user requests, both in real-time and over a certain duration, can be monitored and used to staff the environment with caregivers 104 and further more efficiently distribute the caregivers within the environment. In some embodiments, data analytics module 130 controls which notifications go to which caregiver communication devices 108 and how frequently, as will be discussed in greater detail below. Data analytics module 130 is also effective to monitor trends in the substance of user requests, as well as user satisfaction. This allows an environment, such a hospital, to identify where care is being effectively administered and where care could be more effectively administered.

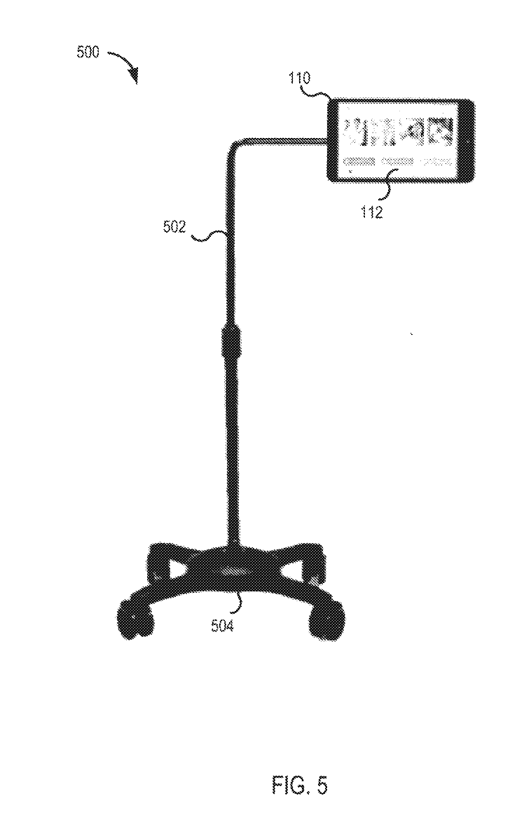

Referring now to FIG. 5, in some embodiments, user communication device 110 is positioned on a stand 500. In some embodiments, user communication device is reversibly mounted on stand 500. In some embodiments, stand 500 includes a reversibly extendable arm 502. In some embodiments, stand 500 and/or arm 502 are mounted to a floor, a ceiling, a wall, a wheelchair, a base, or combinations thereof. In some embodiments, stand 500 includes an alarm configured to sound should the stand fall down or become disconnected, e.g., from a floor, a ceiling, a wall, a wheelchair, a base, etc.

In some embodiments, stand 500 and/or arm 502 are manipulated manually. In some embodiments, the stand 500/arm 502 is motorized to be transformed from a retracted position to an extended, in-use position. In some embodiments, stand 500/arm 502 rotates, translates, extends/folds, or combinations thereof in order to transform from the retracted position to an in-use position for use by user 102. In some embodiments, movement of stand 500/arm 502 is controlled via user communication device 110, via a button press, a voice keyword, or combinations thereof. In some embodiments, the retracted position is against a floor, a ceiling, a wall, a wheelchair, under or within a piece of furniture, etc., out of the way of the user's body and eye line and providing convenient storage, e.g., for a sleep or rest mode. Such motorized embodiments are particularly useful for motor impairment patients (e.g., Parkinson's, with upper limb restraints, muscle weakness, paralysis, etc.) or motion restrictions (e.g., IV placement).

In some embodiments, stand 500 includes a base 504. In some embodiments, base 504 includes a fastener, such as a hook, for attaching/hanging the base with or without retractable arm 502 to a surface for storage, e.g., in a closet. In some embodiments, stand 500 retracts/collapses to base 504 for increased portability. This design provides convenient storage for deep cleaning of rooms and allows caregivers 104 and staff to conveniently store the inactivated stand on the fastener on the wall if wanted.

In some embodiments, base 504 includes a power supply for powering user communication device 110. Thus, if user communication device 110 runs out of power while mobile and disconnected from stand 500, the device can have extended battery life. In some embodiments, base 504 includes a battery powerpack. In some embodiments, power supply is rechargeable. The rechargeable powerpack allows for extended battery life and a means of backup battery in case of a power outage or natural disaster.

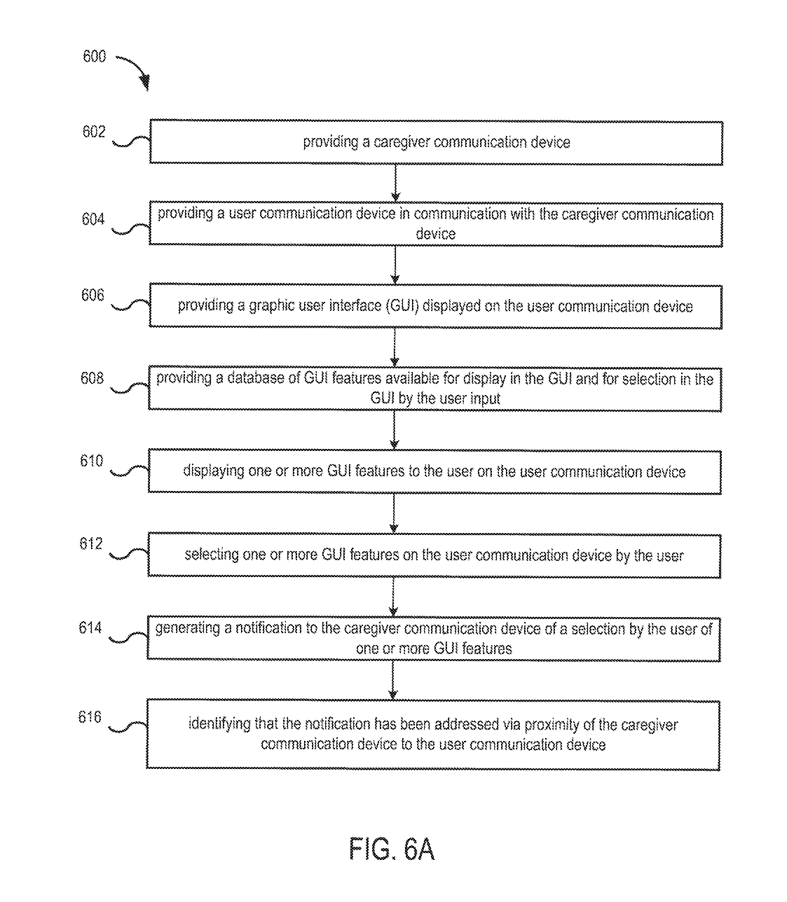

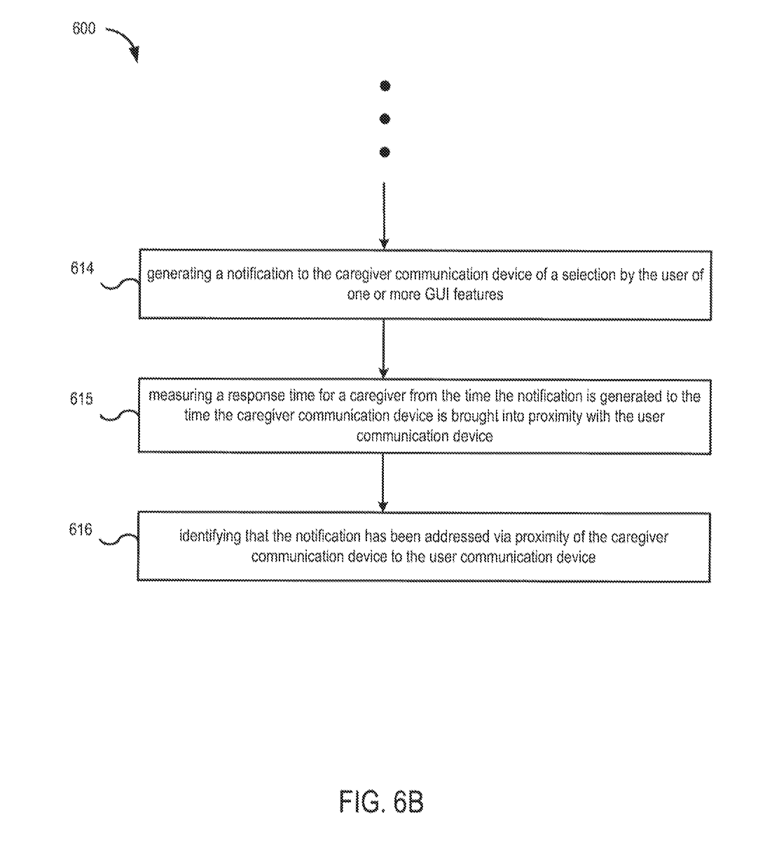

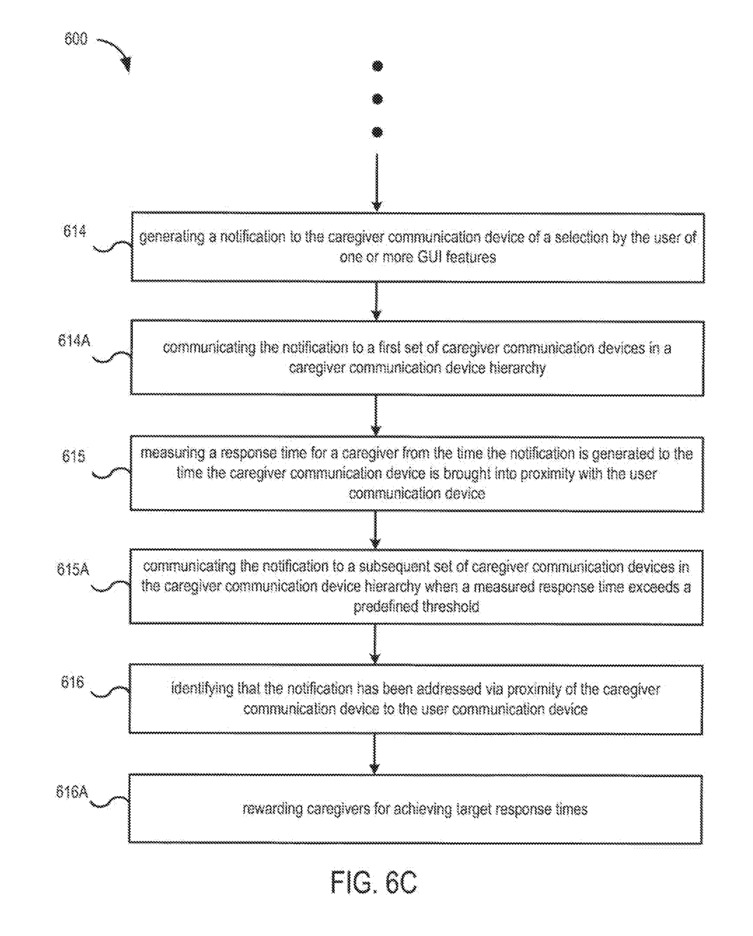

Referring now to FIG. 6A, in some embodiments, some embodiments of the present disclosure are directed to a computer implemented method 600 for improving communication between a user and a caregiver. At 602, a caregiver communication device is provided, e.g., to a nurse, doctor, flight attendant, waiter, etc. At 604, a user communication device is provided in communication with the caregiver communication device, facilitating communication between the user, e.g., a patient, passenger, customer, etc., and the caregiver, as discussed above. At 606, a GUI is provided and displayed on the user communication device. As discussed above, in some embodiments, the GUI includes a graphical cursor configured to identify a location of user input in the GUI. At 608, a database of GUI features available for display in the GUI and for selection in the GUI by the user input is provided. As discussed above, in some embodiments, a user or caregiver/administrator can customize the GUI with GUI features of particular interest and/or use to the user. At 610, the one or more GUI features are displayed to the user on the user communication device. At 612, the user selects one or more GUI features on the user communication device. At 614, a notification is generated to the caregiver communication device of the selection by the user of the one or more GUI features. Having been notified of the request from the user, the caregiver then fulfills the user the request. At 616, the notification is identified as addressed, e.g., via proximity of the caregiver communication device to the user communication device.

Referring now to FIG. 6B, and as discussed above, in some embodiments, at 615 a response time is measured for a caregiver corresponding to, e.g., the time the notification is generated to the time the user request is addressed, e.g., when the caregiver communication device is brought into proximity with the user communication device, when the caregiver communication device scans a request-associated code, etc.

Referring now to FIG. 6C, in some embodiments, at 614A, the notification is communicated to a first set of caregiver communication devices in a caregiver communication device hierarchy. At 615A, the notification is communicated to a subsequent set of caregiver communication devices in the caregiver communication device hierarchy when a measured response time exceeds a predefined threshold. In these embodiments, caregivers are arranged across a plurality of sets, i.e., groups. In some embodiments, the notification is communicated to a first set of caregiver communication devices. In some embodiments, if the notification is not addressed by the first set of caregivers, e.g., exceeds a predetermined response time threshold, the notification is then communicated to a second set of caregiver communication devices. In some embodiments, at least one of the caregivers in the second set is different from the first set of caregivers. In some embodiments, the sets of caregivers/caregiver communication devices are arranged in a hierarchy. In these embodiments, the first set is the initial group of caregivers expected to address the user request. If the first set fails to address the notification, additional sets of caregivers are informed that the request has been sitting unaddressed and care may be needed more urgently. The sets of caregivers in the hierarchy can be arranged in any desired composition. By way of example, the first set of caregivers can include only the nurse specifically assigned to a patient's room. The second set of caregivers could include one or more residents in the building and a number of other nurses known to be working on the floor that day. Additional sets of caregivers might include attending physicians, “all-hands” sets of every caregiver, etc. for particularly neglected requests. In some embodiments, at 616A, caregivers are rewarded for achieving target response times.

Methods and systems of the present disclosure are advantageous in that they provide a flexible system that greatly improves communication between a caregiver, e.g., a doctor, nurse, or other service provider, and a user receiving their care. The GUI of the user communication device can be customized to include selections tailored to user needs and desires. This declutters the GUI of irrelevant material and enables more efficient notification of the caregiver, which can be particularly useful in environments such as hospitals where the speed at which care is administered can be especially important. The network of communication devices also facilitates communication by users who may not be able to communicate verbally, e.g., during recuperation from surgery. The GUI can be tailored specifically for use by children, seniors, those with visual or physical impairments, etc., all to maximize benefits to the user and enable more effective care by the caregiver. The hands-free module is also particularly advantageous for hospital or long-term care implementations as it facilitates communication with disabled or physically impaired patients. Arrangement of caregivers in a notification hierarchy or cascade also helps ensure that a user's request for assistance cannot get lost or forgotten, further promoting attentive handling of the requests. Thus the challenges often faced by caregivers who are assigned to care for a plurality of individuals simultaneously are mitigated.

The systems also include robust data analytics functionality to monitor the effectiveness of the caregivers and the attentiveness received by the users in response to their requests. Trends in user requests can be identified so that appropriate action by the institution implementing the system can be taken, e.g., Gatorade can be identified as a particularly popular choice amongst patients so orders of Gatorade are increased. The data can also be useful in demonstrating the effectiveness of the institution, e.g., for marketing purposes.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A system for improving communication between a user and a caregiver within an environment, comprising:
a caregiver communication device in the environment;
a graphic user interface (GUI) displayed on a user communication device in the environment in communication with the caregiver communication device, the GUI of the user communication device including a graphical cursor configured to identify a location of user input in the GUI;
a database of GUI features available for display in the GUI and for selection in the GUI by the user input;
a non-transitory computer storage media coupled with the user communication device and encoded with one or more computer programs for facilitating communication of the user communication device with the caregiver communication device, the one or more computer programs including:
a GUI customization module, the GUI customization module configured to organize the GUI features in the GUI for presentation to a user via the user communication device and selection by the user via the graphical cursor;
a notification module, the notification module configured to:
transmit a notification to the caregiver communication device of one or more GUI features selected by the user, and
transmit notifications from the caregiver to the user communication device;
and,
a hands-free module, the hands-free module being in communication with the GUI and configured to enable manipulation of the graphical cursor within the GUI without physical contact with the user communication device by the user.

2. The system according to claim 1, wherein the one or more computer programs further comprise:
a translation module configured to receive a GUI feature selection in a first language and output an equivalent notification in a second language.

3. The system according to claim 1, wherein the GUI features include user-needs identifiers related to general user assistance, user bathroom needs, user present status, user mood, user pain level, user pain location, food requests, user satisfaction, predefined messages, environmental controls, or combinations thereof; and
notifications from the caregiver include informational documents, responses to GUI features selected by the user, presence of the caregiver communication device at a predetermined location within the environment or combinations thereof.

4. The system according to claim 1, wherein the notification module includes a messaging module configured to transmit non-GUI feature messages between the caregiver communication device and the user communication device.

5. The system according to claim 1, wherein the notifications include a status indicator corresponding to whether the one or more GUI features selected by the user have been addressed, wherein addressing the one or more GUI features selected by the user includes:
identifying the caregiver communication device at a predetermined location within the environment via Wi-Fi, global positioning system, near-field communication, radio-frequency identification, or combinations thereof;
scanning by the caregiver communication device of a code associated with the one or more GUI features; or
combinations thereof.

6. The system according to claim 5, further comprising one or more wireless beacons configured to track the location of the caregiver communication device within the environment, wherein the status indicator of the one or more GUI features is changed to completed when the caregiver communication device enters a user's room.

7. The system according to claim 1, wherein the hands-free module enables manipulation of the graphical cursor via facial recognition, head-tracking, voice recognition, or combinations thereof.

8. The system according to claim 1, wherein the hands-free module enables selection of a first GUI feature via predetermined overlap duration of the graphical cursor and the first GUI feature in the GUI, and the hands-free module includes a head-tracking module in communication with the GUI, the head-tracking module being configured to manipulate the graphical cursor within the GUI in response to movement of a user's head.

9. The system according to claim 8, wherein the head-tracking module is configured to:
continuously capture frames corresponding to an environment in front of the user communication device;
identifying the presence of a user's face within the frames;
define one or more reference points on the user's face;
position a virtual window enclosing the one or more reference points, the virtual window corresponding to the graphical cursor;
monitor the position of the one or more reference points at a time (t) compared to a prior time (t−x);
determine a flow vector for a reference point corresponding to movement of the reference point from the prior time (t−x) to time (t); and
updating the position of the graphical cursor corresponding to an average of the flow vectors.

10. The system according to claim 9, further comprising:
updating a sensitivity of the head-tracking module to increase or decrease the response of the head-tracking module to movement of the reference point.

11. The system according to claim 1, further comprising one or more user-family communication devices, wherein the notification module is configured to transmit a notification to the one or more user-family communication devices of one or more GUI features selected by the user.

12. A computer implemented method for improving communication between a user and a caregiver, comprising:
providing a caregiver communication device;
providing a user communication device in communication with the caregiver communication device;
providing a graphic user interface (GUI) displayed on the user communication device, the GUI including a graphical cursor configured to identify a location of user input in the GUI;
providing a database of GUI features available for display in the GUI and for selection in the GUI by the user input;
displaying one or more GUI features to the user on the user communication device;
selecting one or more GUI features on the user communication device by the user;
generating a notification to the caregiver communication device of a selection by the user of one or more GUI features; and
identifying that the notification has been addressed via proximity of the caregiver communication device to the user communication device.

13. The method according to claim 12, further comprising:
measuring a response time for a caregiver from the time the notification is generated to the time the caregiver communication device is brought into proximity with the user communication device.

14. The method according to claim 13, further comprising:
communicating the notification to a first set of caregiver communication devices in a caregiver communication device hierarchy; and
communicating the notification to a subsequent set of caregiver communication devices in the caregiver communication device hierarchy when a measured response time exceeds a predefined threshold.

15. The method according to claim 13, further comprising rewarding caregivers for achieving target response times.

16. The method according to claim 12, further comprising
scheduling a change in the GUI features selectable on the user communication device by the user for a predetermined instance; and
changing the GUI features selectable on the user communication device at the predetermined instance.

17. The method according to claim 12, wherein the GUI features include user-needs identifiers related to general user assistance, user bathroom needs, user present status, user mood, user pain level, user pain location, food requests, user satisfaction, predefined messages, or combinations thereof.

18. The method according to claim 12, wherein selecting by the user one or more GUI features is performed via a head-tracking module in communication with the GUI, the head-tracking module being configured to manipulate the graphical cursor within the GUI in response to movement of a user's head.

19. The method according to claim 18, wherein the head-tracking module is configured to:
continuously capture frames corresponding to an environment in front of the user communication device;
identifying the presence of a user's face within the frames;
define one or more reference points on the user's face;
position a virtual window enclosing the one or more reference points, the virtual window corresponding to the graphical cursor;
monitor the position of the one or more reference points at a time (t) compared to a prior time (t−x);
determine a flow vector for a reference point corresponding to movement of the reference point from the prior time (t−x) to time (t); and
updating the position of the graphical cursor corresponding to an average of the flow vectors.

20. A system for improving communication between a user and a caregiver within an environment, comprising:
a wireless communication network;
a caregiver communication device in communication with the wireless communication network;
a user communication device in communication with the caregiver communication device via the wireless communication network;
a graphic user interface (GUI) displayed on the user communication device, the GUI including a graphical cursor configured to identify a location of user input in the GUI;
a database of GUI features available for display in the GUI and for selection in the GUI by the user input;
a non-transitory computer storage media coupled with the user communication device and encoded with one or more computer programs for facilitating communication of the user communication device with the caregiver communication device, the one or more computer programs including:
a GUI customization module, the GUI customization module configured to organize the GUI features in the GUI for presentation to a user via the user communication device and selection by the user via the graphical cursor;
a notification module, the notification module configured to:
transmit a notification to the caregiver communication device of one or more GUI features selected by the user,
transmit notifications from the caregiver communication device to the user communication device; and
transmit notifications from the user communication device and the caregiver communication device to an electronic medical record for the user;
a head-tracking module, the head-tracking module being in communication with the GUI and configured to enable manipulation of the graphical cursor within the GUI via movement of the user's head within an environment in front of the user communication device;
a caregiver tracking module, the caregiver tracking module configured to track the location of a caregiver in the environment and identify when the caregiver is in proximity with the user; and
a data analytics module, the data analytics module configured to record data regarding the caregiver communication device, the user communication device, the GUI features, the transmitted notifications, the hands-free-module, or combinations thereof;
and
a stand upon which the user communication device is reversibly mounted, the stand including a reversibly extendable arm mounted to a floor, a ceiling, a wall, a wheelchair, a base, or combinations thereof,
wherein movement of the reversibly extendable arm is controlled via the user communication device.
